(19) United States
(12) Patent Application Publication
(10) Pub. No.: US 2016/0136645 A1
(43) Pub. Date: May 19, 2016

(54) CONTAINER STORAGE RECEPTACLE

(71) Applicant: Seiko Epson Corporation, Tokyo (JP)

(72) Inventors: Kotaro IDEGAMI, Chino (JP); Masato HANAMURA, Shiojiri (JP)

(21) Appl. No.: 14/942,162

(22) Filed: Nov. 16, 2015

(30) Foreign Application Priority Data
Nov. 17, 2014 (JP) 2014-232505

(51) Int. Cl. B01L 3/00; B65D 77/04; C12Q 1/68; B65D 65/38

(52) U.S. Cl. CPC B01L 3/52; B65D 65/38; B65D 77/0406; C12Q 1/6806; B01L 2300/0832; B01L 2200/16

(57) ABSTRACT

A container storage receptacle includes a package and an elution container that is enclosed and stored in the package, and that encloses and stores an eluent for eluting a nucleic acid from a nucleic acid binding activity solid-phase carrier. The eluent contains water, and an inner portion of the package is in a state of being saturated with water vapor.

### BACKGROUND

1. Technical Field

The present invention relates to a container storage receptacle.

2. Related Art

In the field of biochemistry, a technology relating to polymerase chain reaction (PCR) has been established. Recently, amplification accuracy or detection sensitivity has been improved in a PCR method, thereby enabling a very minor amount of a specimen (DNA or the like) to be amplified, detected, and analyzed. The PCR is a technique of amplifying a target nucleic acid by applying a thermal cycle to a solution (reaction solution) containing a nucleic acid of an amplification target (target nucleic acid) and a reagent. As the thermal cycle in the PCR, a technique of applying the thermal cycle at two or three stage temperature is generally used.

On the other hand, at present, a simple test kit such as an immuno-chromatograph kit is mainly used in order to diagnose infectious diseases such as influenza in the medical field. However, such a simple test does not ensure sufficiently accurate diagnosis in some cases. Accordingly, in order to diagnose infectious diseases, it is desired to employ the PCR by which higher test accuracy can be expected.

In recent years, as a device using the PCR method, a device has been proposed which purifies a nucleic acid by alternately stacking an aqueous liquid layer and a non-water soluble gel layer in a capillary (in a cartridge), and by causing magnetic particles having the attached nucleic acid to pass therethrough (refer to International Publication No. 2012/086243). International Publication No. 2012/086243 discloses that alcohol is used as a cleaning solution for cleaning the magnetic particle to which the nucleic acid adheres, and water is used as an elution solution for eluting the nucleic acid from the magnetic particle.

However, according to the above-described device, for example, in some cases of long-term storage, an eluent inside an elution container evaporates, thereby decreasing the amount of the eluent. Consequently, in some cases, PCR is adversely affected.

### SUMMARY

An advantage of some aspects of the invention is to provide a container storage receptacle which can prevent an eluent from evaporating even in a case of long-term storage.

### APPLICATION EXAMPLE 1

A container storage receptacle according to this application example includes a package, and an elution container that is enclosed and stored in the package, and that encloses and stores an eluent for eluting a nucleic acid from a nucleic acid binding activity solid-phase carrier. The eluent contains water, and an inner portion of the package is in a state of being saturated with water vapor.

According to the container storage receptacle of this application example, the water contained in the eluent inside the elution container can be prevented from evaporating after permeating through the elution container and the package. Therefore, according to the container storage receptacle in the application example, even in a case of long-term storage, the eluent can be prevented from evaporating.

### APPLICATION EXAMPLE 2

The container storage receptacle according to the application example may further include a liquid holding member that is enclosed and stored in the package, and that contains water.

According to the container storage receptacle of this application example, without infusing liquid water into the package, the inner portion of the package can be brought into a state of being saturated with the water vapor. For example, if the liquid water is infused into the package, when an elution container is detached from the package, the water spills therefrom in some cases. Therefore, according to the container storage receptacle in the application example, it is not necessary to infuse the liquid water into the package. Accordingly, when the elution container is detached from the package, there is no possibility that the water may spill therefrom.

### APPLICATION EXAMPLE 3

In the container storage receptacle according to the application example, the liquid holding member may be absorbent cotton.

According to the container storage receptacle of this application example, even in a case of long-term storage, the elution can be prevented from evaporating.

### APPLICATION EXAMPLE 4

In the container storage receptacle according to the application example, water permeability of the package may be lower than water permeability of the elution container.

According to the container storage receptacle of this application example, the water contained in the eluent inside the elution container can be more reliably prevented from evaporating after permeating through the elution container and the package.

### APPLICATION EXAMPLE 5

In the container storage receptacle according to the application example, a fluid which is immiscible with the eluent may be enclosed and contained in the elution container.

According to the container storage receptacle of this application example, the eluent can employ a plug shape.

### APPLICATION EXAMPLE 6

The container storage receptacle according to the application example may further include a cleaning container that is enclosed and stored in the package, and that encloses and stores a cleaning solution for cleaning a nucleic acid binding activity solid-phase carrier having the adsorbed nucleic acid. The cleaning solution contains the water, and water permeability of the package may be lower than water permeability of the cleaning container.

According to the container storage receptacle of this application example, the water contained in the cleaning solution inside the cleaning container can be prevented from evaporating after permeating through the package.

### APPLICATION EXAMPLE 7

In the container storage receptacle according to the application example, the package may be a bag which has an aluminum layer.

According to the container storage receptacle of this application example, the water permeability of the package can be lowered by the aluminum layer.

### BRIEF DESCRIPTION OF THE DRAWINGS

The invention will be described with reference to the accompanying drawings, wherein like numbers reference like elements.

FIG. 1 is a front view of a container assembly according to an embodiment.

FIG. 2 is a side view of the container assembly according to the embodiment.

FIG. 3 is a plan view of the container assembly according to the embodiment.

FIG. 4 is a perspective view of the container assembly according to the embodiment.

FIG. 5 is a sectional view of the container assembly according to the embodiment, which is taken along line A-A in FIG. 3.

FIG. 6 is a sectional view of the container assembly according to the embodiment, which is taken along line C-C in FIG. 3.

FIGS. 7A and 7B are schematic views for describing an operation of the container assembly according to the embodiment.

FIGS. 8A and 8B are schematic views for describing an operation of the container assembly according to the embodiment.

FIG. 9 is a schematic configuration diagram of a PCR device.

FIG. 10 is a block diagram of the PCR device.

FIG. 11 is a sectional view of a cartridge set according to the embodiment.

FIG. 12 is a sectional view of a first package according to the embodiment.

FIG. 13 is a sectional view of a first temporarily assembled body according to the embodiment.

FIG. 14 is a sectional view of a second temporarily assembled body according to the embodiment.

FIG. 15 is a sectional view of a reaction container according to the embodiment.

FIG. 16 is a sectional view of a cartridge set according to the embodiment.

FIG. 17 is a sectional view of a cartridge set according to a modification example.

### DESCRIPTION OF EXEMPLARY EMBODIMENTS

Hereinafter, a preferred embodiment according to the invention will be described in detail with reference to the drawings. The embodiment described below does not unduly limit the content of the invention described in the appended claims. In addition, all configuration elements described below are not necessarily indispensable configuration requirements for the invention.

A cartridge set according to the invention is used in assembling a cartridge for performing PCR. That is, the cartridge for performing the PCR can be obtained by assembling the cartridge set. Hereinafter, the cartridge (container assembly) will be described, and then the cartridge set will be described.

### 1. Overview of Container Assembly

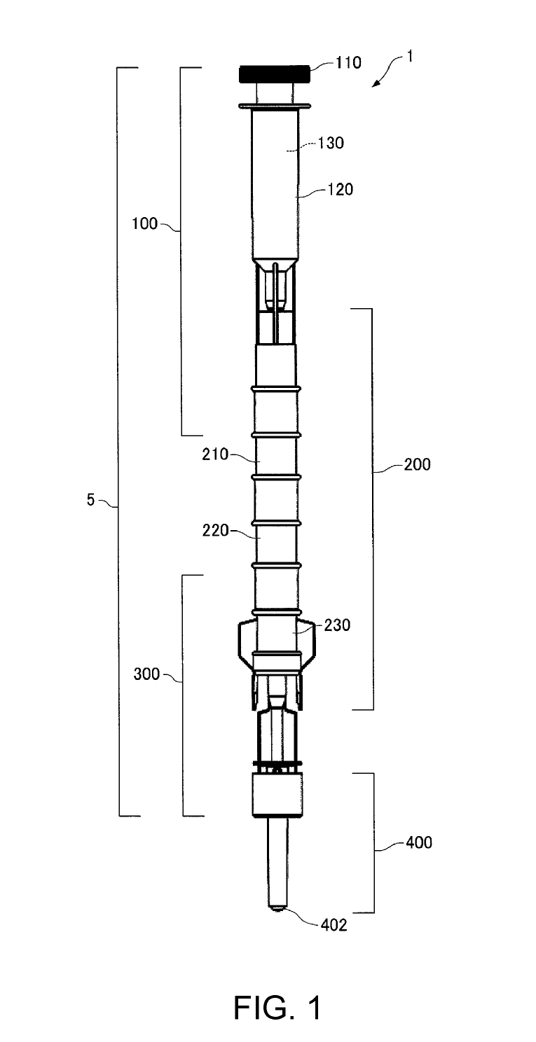

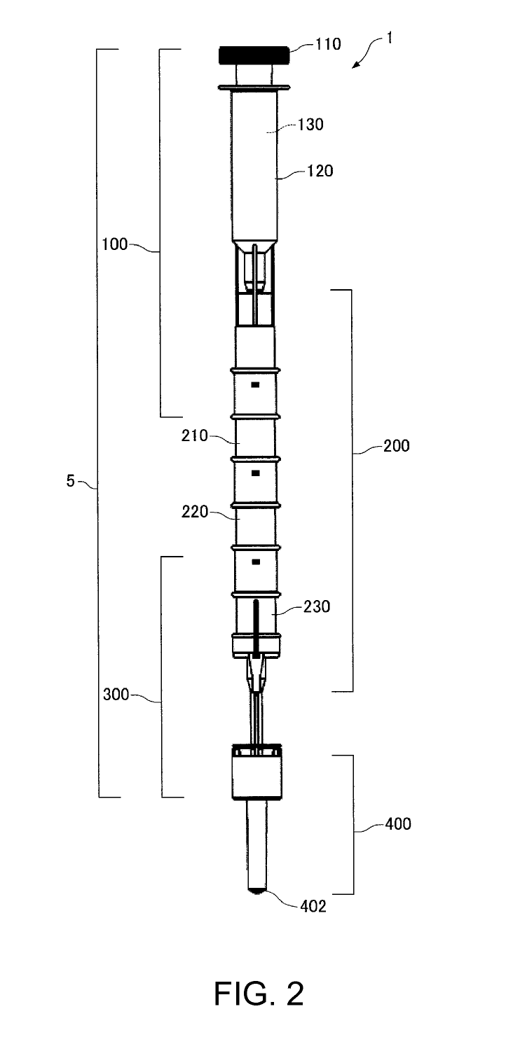

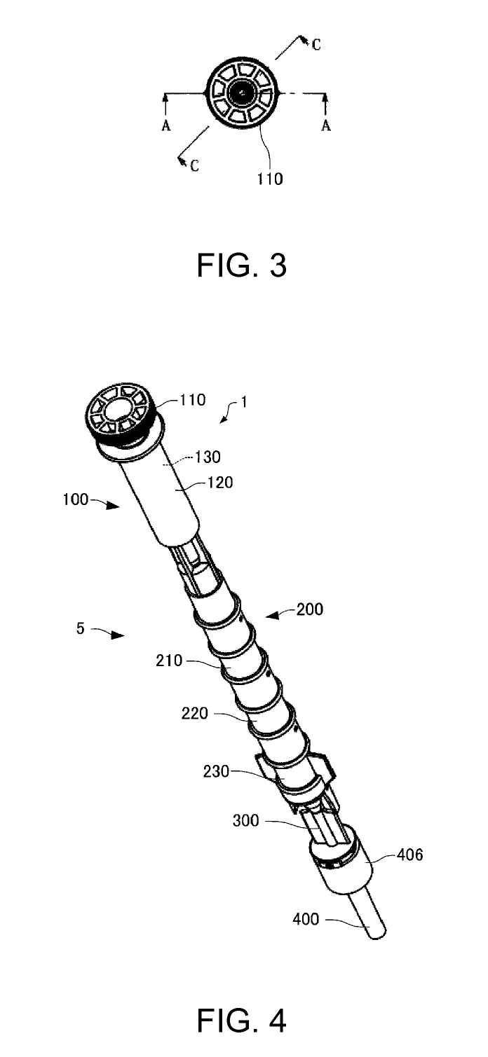

An overview of a container assembly 1 according to the present embodiment will be described with reference to FIGS. 1 to 4. FIG. 1 is a front view of the container assembly 1 (hereinafter, sometimes referred to as a cartridge) according to the embodiment. FIG. 2 is a side view of the container assembly 1 according to the embodiment. FIG. 3 is a plan view of the container assembly 1 according to the embodiment. FIG. 4 is a perspective view of the container assembly 1 according to the embodiment. Description will be made on the assumption that a state of the container assembly 1 illustrated in FIGS. 1 to 3 represents an upright state.

The container assembly 1 includes an adsorption container 100, a cleaning container 200, an elution container 300, and a reaction container 400. The container assembly 1 configures a flow path which allows communication from the adsorption container 100 to the reaction container 400. In the flow path of the container assembly 1, one end portion thereof is closed by a cap 110, and the other end portion is closed by a bottom portion 402.

The container assembly 1 binds a nucleic acid with magnetic beads (not illustrated) inside the adsorption container 100, and purifies the nucleic acid while the magnetic beads move in the cleaning container 200. The container assembly 1 performs preprocessing for eluting the nucleic acid into an eluent droplet (not illustrated) inside the elution container 300, and performs thermal cycle processing for a polymerase reaction on the eluent droplet containing the nucleic acid inside the reaction container 400.

A material of the container assembly 1 is not particularly limited. However, for example, glass, a polymer, or metal can be used. As the material of the container assembly 1, it is preferable to select a transparent material invisible light such as the glass or the polymer, since an inner portion (inside of a cavity) thereof can be observed from the outside of the container assembly 1. As the material of the container assembly 1, it is preferable to select a magnetic force transmitting material or a non-magnetic material, since the magnetic beads (not illustrated) can be easily caused to pass through the container assembly 1 by applying a magnetic force to the container assembly 1 from the outside of the container assembly 1. For example, the material of the container assembly 1 can employ a polypropylene resin.

The inner portion of the adsorption container 100 has a cylindrical syringe portion 120 for containing an adsorption solution, a plunger portion 130 functioning as a movable plunger inserted into the syringe portion 120, and the cap 110 fixed to one end portion of the plunger portion 130. The adsorption container 100 causes the plunger portion 130 to slide on an inner surface of the syringe portion 120 by moving the cap 110 toward the syringe portion 120. In this manner, an adsorption solution (not illustrated) contained inside the syringe portion 120 can be pressed toward the cleaning container 200. The adsorption solution will be described later.

The cleaning container 200 can be obtained by joining and assembling first to third cleaning containers 210, 220, and 230. Each inner portion of the first to third cleaning containers 210, 220, and 230 has one or more cleaning solution layers which are divided by an oil layer (not illustrated). Then, the first to third cleaning containers 210, 220, and 230 are joined to each other, thereby allowing the inner portion of the cleaning container 200 to have multiple cleaning solution layers which are divided by multiple oil layers (not illustrated). In the cleaning container 200 according to the embodiment, an example has been described which employs three cleaning containers including the first to third cleaning containers 210, 220, and 230. However, without being limited thereto, the number of cleaning containers can be increased or decreased depending on the number of cleaning solution layers. A cleaning solution will be described later.

The elution container 300 is joined to the third cleaning container 230 of the cleaning container 200, and an inner portion thereof contains an eluent so that a shape of a plug can be maintained. Here, the “plug” means a specific liquid when the specific liquid occupies one division inside a flow path. More specifically, the plug of the specific liquid indicates a columnar liquid in which only the specific liquid substantially occupies the inner portion in a longitudinal direction of the flow path, and represents a state where a certain space of the inner portion in the flow path is divided by the plug of the liquid. Here, the expression of “substantially” indicates that a small amount (for example, like a thin film) of other substances (liquid or the like) may be present around the plug, that is, on an inner wall of the flow path. The eluent will be described later.

A nucleic acid purification device 5 includes the adsorption container 100, the cleaning container 200, and the elution container 300.

The reaction container 400 is joined to the elution container 300, and receives the liquid pressed out from the elution container 300. The reaction container 400 contains a droplet of the eluent containing a specimen during thermal cycle processing. The reaction container 400 contains a reagent (not illustrated). The reagent will be described later.

### 2. Detailed Structure of Container Assembly

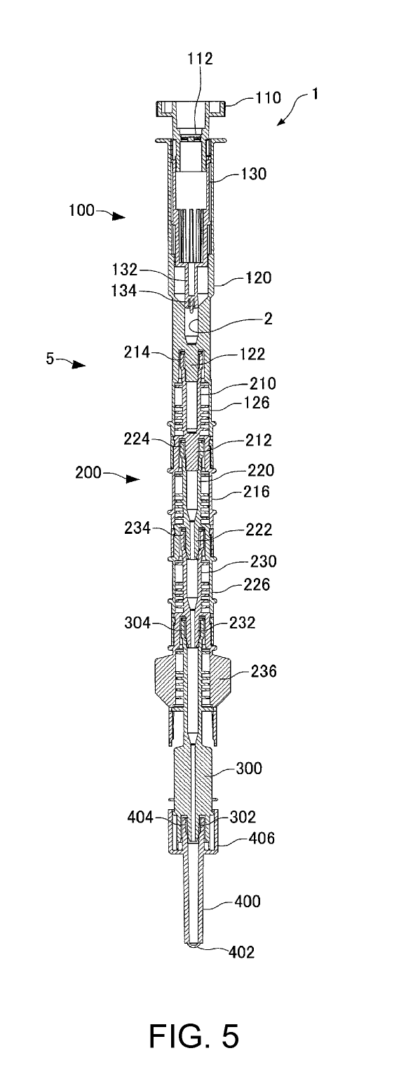

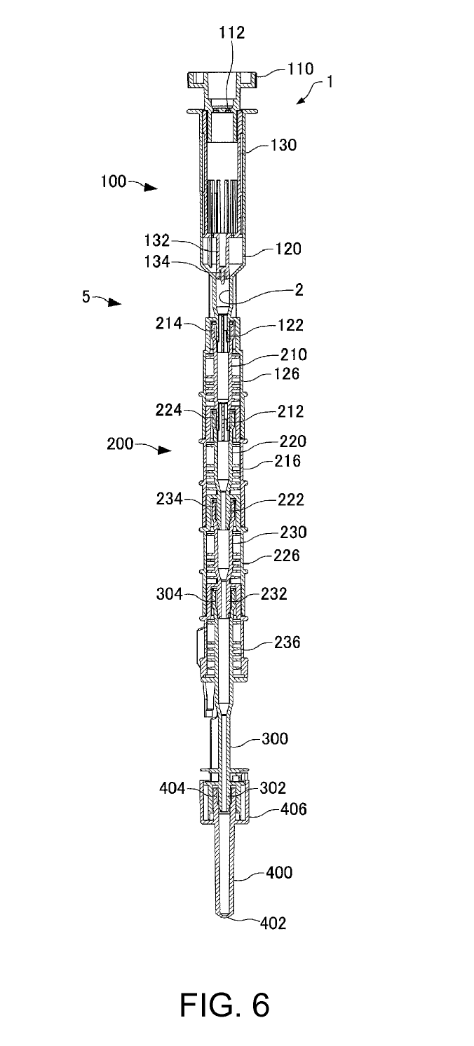

Next, a detailed structure of the container assembly 1 will be described with reference to FIGS. 5 and 6. FIG. 5 is a sectional view of the container assembly 1 according to the embodiment, which is taken along line A-A in FIG. 3. FIG. 6 is a sectional view of the container assembly 1 according to the embodiment, which is taken along line C-C in FIG. 3. In practice, the container assembly 1 is assembled in a state of being filled with content such as the cleaning solution. However, since FIGS. 5 and 6 are views for describing the structure of the container assembly 1, the illustration of the content is omitted.

### 2-1. Adsorption Container

The plunger portion 130 is inserted into the adsorption container 100 through one opening end portion of the syringe portion 120, and the cap 110 is inserted into an opening end portion of the plunger portion 130. The cap 110 has a vent 112 at the center thereof, and the vent 112 can suppress a change in internal pressure of the plunger portion 130 when the plunger portion 130 is operated.

The plunger portion 130 is a substantially cylindrical plunger which slides on an inner peripheral surface of the syringe portion 120, and has the opening end portion into which the cap 110 is inserted, a rod-shaped portion 132 which extends in the longitudinal direction of the syringe portion 120 from a bottom portion facing the opening end portion, and a distal end portion 134 at a distal end of the rod-shaped portion 132. The rod-shaped portion 132 protrudes from the center of the bottom portion of the plunger portion 130. A through-hole is formed around the rod-shaped portion 132, thereby allowing the inside of the plunger portion 130 and the inside of the syringe portion 120 to communicate with each other.

The syringe portion 120 configures a portion of a flow path 2 in the container assembly 1, and has a large diameter portion for containing the plunger portion 130, a small diameter portion whose inner diameter is smaller than that of the large diameter portion, a diameter reduced portion whose inner diameter is reduced from the large diameter portion toward the small diameter portion, an adsorption/insertion portion 122 disposed at a distal end of the small diameter portion, and a cylindrical adsorption cover portion 126 for covering the periphery of the adsorption/insertion portion 122. The large diameter portion, the small diameter portion, and the adsorption/insertion portion 122 which configure a portion of the flow path 2 in the container assembly 1 have a substantially cylindrical shape.

When being provided for a worker, a distal end portion 134 of the plunger portion 130 forms two divisions by enclosing the small diameter portion of the syringe portion 120 and by being divided into the large diameter portion, the diameter reduced portion, and the small diameter portion.

The adsorption/insertion portion 122 of the syringe portion 120 is inserted and fitted into a first receiving portion 214 which is one opening end portion of the first cleaning container 210 in the cleaning container 200, thereby joining the syringe portion 120 and the first cleaning container 210 to each other. An outer peripheral surface of the adsorption/insertion portion 122 and an inner peripheral surface of the first receiving portion 214 come into close contact with each other, thereby preventing the liquid as the content from leaking outward.

### 2-2. Cleaning Container

The cleaning container 200 configures a portion of the flow path 2 in the container assembly 1, and is an assembly including the first to third cleaning containers 210, 220, and 230. The first to third cleaning containers 210, 220, and 230 have the same basic structure. Accordingly, the structure of the first cleaning container 210 will be described, and description of the second and third cleaning containers 220 and 230 will be omitted.

The first cleaning container 210 has a substantially cylindrical shape which extends in the longitudinal direction of the container assembly 1, and has a first insertion portion 212 which is formed in one opening end portion, the first receiving portion 214 which is formed in the other opening end portion, and a first cylindrical cover portion 216 which covers the periphery of the first insertion portion 212.

The outer diameter of the first insertion portion 212 is substantially the same as the inner diameter of a second receiving portion 224. The inner diameter of the first receiving portion 214 is substantially the same as the outer diameter of the adsorption/insertion portion 122.

The first insertion portion 212 of the first cleaning container 210 is inserted and fitted into the second receiving portion 224 of the second cleaning container 220. In this manner, the outer periphery of the first insertion portion 212 comes into close contact with the inner periphery of the second receiving portion 224, thereby sealing and joining the first cleaning container 210 and the second cleaning container 220 to each other. Similarly, the first to third cleaning containers 210, 220, and 230 are connected to each another, thereby forming the cleaning container 200. Here, the “sealing” means enclosing at least for preventing a liquid or gas contained in the container from leaking out, and may include enclosing for preventing a liquid or gas from permeating through the inner portion from the outside.

### 2-3 Elution Container

The elution container 300 has a substantially cylindrical shape which extends in the longitudinal direction of the container assembly 1, and configures a portion of the flow path 2 in the container assembly 1. The elution container 300 has an elution/insertion portion 302 formed in one opening end portion, and an elution receiving portion 304 formed in the other opening end portion.

The inner diameter of the elution receiving portion 304 is substantially the same as the outer diameter of a third insertion portion 232 of the third cleaning container 230. The third insertion portion 232 is inserted and fitted into the elution receiving portion 304. In this manner, the outer periphery of the third insertion portion 232 comes into close contact with the inner periphery of the elution receiving portion 304, thereby sealing and joining the third cleaning container 230 and the elution container 300 to each other.

### 2-4. Reaction Container

The reaction container 400 has a substantially cylindrical shape which extends in the longitudinal direction of the container assembly 1, and configures a portion of the flow path 2 in the container assembly 1. The reaction container 400 has a reaction receiving portion 404 formed in one opening end portion, a bottom portion 402 formed in the other closed end portion, and a reservoir portion 406 for covering the reaction receiving portion 404.

The inner diameter of the reaction receiving portion 404 is substantially the same as the outer diameter of the elution/insertion portion 302 of the elution container 300. The elution/insertion portion 302 is inserted and fitted into the reaction receiving portion 404, thereby joining the elution container 300 and the reaction container 400 to each other.

The reservoir portion 406 having a predetermined space is disposed around the reaction receiving portion 404. The reservoir portion 406 has a volume which can contain the liquid spilling from the reaction container 400 due to the movement of the plunger portion 130.

### 3. Content of Container Assembly and Operation of Container Assembly

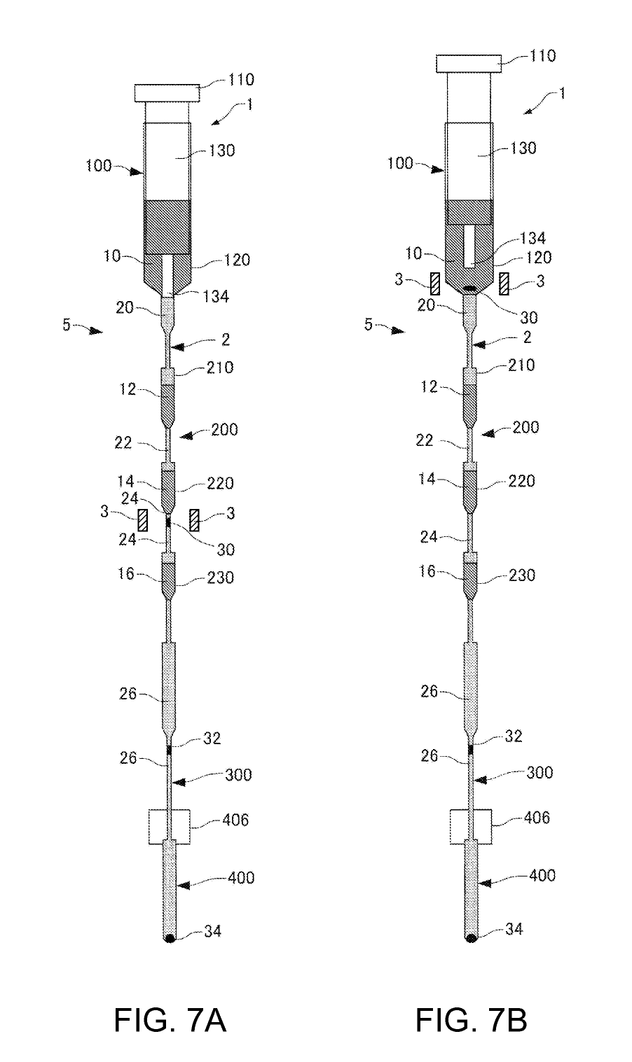

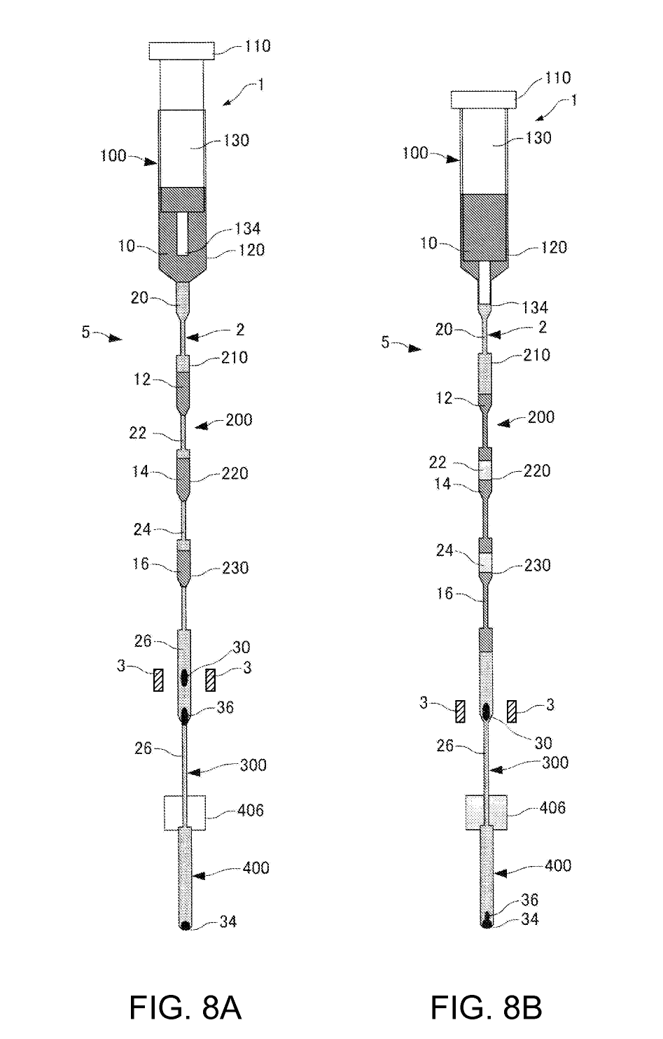

Next, content of the container assembly 1 will be described with reference to FIG. 7A, and an operation of the container assembly 1 will be described with reference to FIGS. 7A to 8B. FIGS. 7A and 7B are schematic views for describing the operation of the container assembly 1 according to the embodiment. FIGS. 8A and 8B are schematic views for describing the operation of the container assembly 1 according to the embodiment. In FIGS. 7A to 8B, each container is expressed by the flow path 2 in order to describe a state of the content, and an outer shape or a joining structure is omitted.

### 3-1. Content

FIG. 7A illustrates a state of the content inside the flow path 2 in the state illustrated in FIG. 1. The content inside the flow path 2 represents an adsorption solution 10, first oil 20, a first cleaning solution 12, second oil 22, a second cleaning solution 14, third oil 24, magnetic beads 30, the third oil 24, a third cleaning solution 16, fourth oil 26, an eluent 32, the fourth oil 26, and a reagent 34, sequentially from the cap 110 toward the reaction container 400.

In the flow path 2, a portion having a larger sectional area of a surface orthogonal to the longitudinal direction of the container assembly 1 (thick portion of the flow path 2) and a portion having a smaller sectional area (thin portion of the flow path 2) are alternately arranged. In a case of the first to fourth oil 20, 22, 24, and 26 and the eluent 32, each is partially or entirely contained in the thin portion of the flow path 2. The sectional area of the thin portion of the flow path 2 has an area capable of stably maintaining an interface therebetween, when the interface between adjacent liquids (may be fluids, the same in the following) which are immiscible with each other is arranged in the thin portion of the flow path 2. Therefore, depending on a liquid arranged in the thin portion of the flow path 2, it is possible to stably maintain an arrangement relationship between the liquid and the other liquid arranged above or below the liquid. In addition, even when an interface between the liquid arranged in the thin portion of the flow path 2 and the other liquid arranged in the thick portion of the flow path 2 is formed in the thick portion of the flow path 2, even if the interface is disturbed by a strong impact, the interface can be stably formed at a predetermined position by the interface being placed in a stationary state.

The thin portion of the flow path 2 is formed inside the adsorption/insertion portion 122, the first insertion portion 212, the second insertion portion 222, the third insertion portion 232, and the elution/insertion portion 302, and extends upward beyond the elution/insertion portion 302 in the elution container 300. The liquid contained in the thin portion of the flow path 2 is stably maintained even before the container is assembled.

### 3-1-1. Oil

Any one of the first to fourth oil 20, 22, 24, and 26 includes oil, and is present as a plug between liquids before and behind each oil in a state illustrated in FIGS. 7A and 7B. In order that the first to fourth oil 20, 22, 24, and 26 are present as the plug, liquids of mutual phase separation, that is, liquids which are immiscible with each other are selected as the adjacent liquids before and behind each oil. The oil configuring the first to fourth oil 20, 22, 24, and 26 may be mutually different types of oil. For example, the oil which can be used therefor can include silicone oil such as dimethyl silicone oil, paraffin oil, mineral oil, and one type selected from a mixture thereof.

### 3-1-2. Adsorption Solution

The adsorption solution 10 indicates a liquid obtained when a nucleic acid is adsorbed by the magnetic beads 30, and for example, is a water solution containing a chaotropic substance. For example, as the adsorption solution 10, it is possible to use 5M guanidine thiocyanate, 2% Triton X-100, and 50 mM Tris-HCl (pH 7.2). The adsorption solution 10 is not particularly limited as long as the adsorption solution 10 contains the chaotropic substance. However, the adsorption solution 10 may contain a surfactant in order to destroy cell membranes or to denature proteins contained in cells. The surfactant is not particularly limited as long as the surfactant is generally used in extracting a nucleic acid from the cells. Specifically, the surfactant includes triton series surfactant such as trition-X, nonionic surfactant such as Tween series surfactant including Tween 20, or anionic surfactant such as sodium N-lauroylsarcosinate sarkosyl (SDS). In particular, it is preferable to use the nonionic surfactant in a range of 0.1% to 2%. Furthermore, preferably the surfactant contains a reducing agent such as 2-mercaptoethanol or dithiothreitol. The solution may be a buffer solution, but preferably shows a neutrality of pH 6 to pH 8. In view of these characteristics, it is preferable that the surfactant specifically contains guanidine salt of 3M to 7M, nonionic surfactant of 0% to 5%, EDTA of 0 mM to 0.2 mM, and a reducing agent of 0 M to 0.2 M.

Here, the chaotropic substance is not particularly limited as long as the chaotropic substance is helpful for a solid-phase carrier to adsorb a nucleic acid while generating a chaotropic ion (monovalent anion whose ionic radius is large) in the water solution and having an effect of increasing water solubility of hydrophobic molecules. Specifically, the chaotropic substance includes guanidine hydrochloride, sodium iodide, and sodium perchlorate. However, among these materials, it is preferable to use guanidine thiocyanate or guanidine hydrochloride which has a strong effect of denaturing proteins. The concentration of these chaotropic substances varies depending on each substance. For example, when guanidine thiocyanate is used, if guanidine hydrochloride is used in a range of 3 M to 5.5M, it is preferable to use guanidine hydrochloride in a range of 5 M or greater.

Since the chaotropic substance is present in the water solution, the nucleic acid is thermodynamically advantageous when the nucleic acid is present while being adsorbed into a solid, compared to when the nucleic acid is present while being surrounded with water. Accordingly, the nucleic acid is adsorbed into a surface of the magnetic beads 30.

### 3-1-3. Cleaning Solution

The first to third cleaning solutions 12, 14, and 16 are used in cleaning the magnetic beads 30 having the nucleic acid bound thereto.

The first cleaning solution 12 is a liquid used in phase separation for both the first oil 20 and the second oil 22. Preferably, the first cleaning solution 12 is water or a low salt concentration water solution. In a case of the low salt concentration water solution, the first cleaning solution 12 is preferably a buffer solution. Preferably, the salt concentration of the low salt concentration water solution is 100 mM or smaller, more preferably 50 mM or smaller, and most preferably 10 mM or smaller. The first cleaning solution 12 may contain the above-described surfactant, and pH is not particularly limited. The salt for causing the first cleaning solution 12 to function as the buffer solution is not particularly limited, but preferably is salt such as Tris, Hepes, Pipes, and phosphoric acid. Furthermore, preferably, the first cleaning solution 12 contains only an amount which does not inhibit a nucleic acid carrier from adsorbing alcohol, or which does not inhibit a reverse transcription reaction or PCR reaction. In this case, alcohol concentration is not particularly limited.

The first cleaning solution 12 may contain the chaotropic substance. For example, if the first cleaning solution 12 contains guanidine hydrochloride, the magnetic beads 30 can be cleaned while an adsorption state where the nucleic acid is adsorbed into the magnetic beads 30 is maintained or intensified.

The second cleaning solution 14 is a liquid used in phase separation for both the second oil 22 and the third oil 24. Basically, the second cleaning solution 14 may have a composition which is the same as or different from that of the first cleaning solution 12. However, preferably, the second cleaning solution 14 does not substantially contain the chaotropic substance. The reason is to eliminate a possibility that the chaotropic substance may be delivered to the subsequent solution. For example, the second cleaning solution 14 may be formed of a hydrochloric acid buffer solution of 5 mM Tris. As described above, preferably, the second cleaning solution 14 contains alcohol.

The third cleaning solution 16 is a liquid used in phase separation for both the third oil 24 and the fourth oil 26. Basically, the third cleaning solution 16 may have a composition which is the same as or different from that of the second cleaning solution 14. However, the third cleaning solution 16 does not contain alcohol. The third cleaning solution 16 can contain citric acid in order to prevent alcohol from being delivered to the reaction container 400.

### 3-1-4. Magnetic Beads

The magnetic beads 30 adsorb the nucleic acid. Preferably, the magnetic beads 30 have relatively strong magnetic properties so as to be movable by the magnet 3 located outside the container assembly 1. For example, the magnetic beads 30 may be silica breads or silica-coated beads. Preferably, the magnetic beads 30 may be the silica-coated beads.

### 3-1-5. Eluent

The eluent 32 is a liquid used in phase separation for the fourth oil 26, and is present as a plug interposed between the fourth oil 26 and 26 inside the flow path 2 in the elution container 300. The eluent 32 causes the nucleic acid adsorbed into the magnetic beads 30 to be eluted from the magnetic beads 30 to the eluent 32. The eluent 32 is changed to a droplet in the fourth oil 26 by being heated. For example, as the eluent 32, pure water can be used. Here, the “droplet” means a liquid which is surrounded with free surfaces.

### 3-1-6. Reagent

The reagent 34 contains a component required for a reaction. When the reaction in the reaction container 400 shows PCR, the reagent 34 can contain at least one of an enzyme and a primer (nucleic acid) such as DNA polymerase for amplifying a target nucleic acid (DNA) eluted in an eluent droplet 36 (refer to FIGS. 8A and 8B) and a fluorescent probe for detecting amplified products. Here, the reagent 34 contains all of the primer, the enzyme, and the fluorescent probe. The reagent 34 is not compatible with the fourth oil 26, and reacts by being melted when coming into contact with the droplet 36 of the eluent 32 containing the nucleic acid. The reagent 34 is present in a solid state in the lowest region in the direction of gravity of the flow path 2 inside the reaction container 400. For example, as the reagent 34, a lyophilized (freeze-dried) reagent can be used.

### 3-2. Operation of Container Assembly

An operation example of the container assembly 1 will be described with reference to FIGS. 7A to 8B.

The operation of the container assembly 1 includes:

(A) a process of assembling the container assembly by joining the adsorption container 100, the cleaning container 200, the elution container 300, and the reaction container 400,

(B) a process of introducing a specimen containing the nucleic acid into the adsorption container 100 containing the adsorption solution 10,

(C) a process of moving the magnetic beads 30 to the adsorption container 100 from the second cleaning container 220,

(D) a process of causing the magnetic beads 30 to adsorb the nucleic acid by shaking the adsorption container 100,

(E) a process of moving the magnetic beads 30 having the adsorbed nucleic acid to the elution container 300 from the adsorption container 100 after sequentially passing through the first oil 20, the first cleaning solution 12, the second oil 22, the second cleaning solution 14, the third oil 24, the third cleaning solution 16, and the fourth oil 26,

(F) a process of eluting the nucleic acid to the eluent 32 from the magnetic beads 30 inside the elution container 300, and

(G) a process of bringing a droplet containing the nucleic acid into contact with the reagent 34 inside the reaction container 400.

Hereinafter, the respective processes will be sequentially described.

### (A) Process of Assembling Container Assembly 1

As illustrated in FIG. 7A, in the assembling process, the containers from the adsorption container 100 to the reaction container 400 are joined, and the container assembly 1 is assembled so as to form the flow path 2 which is continuous from the adsorption container 100 to the reaction container 400. In FIG. 7A, the cap 110 is mounted on the adsorption container 100. However, the cap 110 is mounted on the plunger portion 130 after the process (B).

More specifically, the elution/insertion portion 302 of the elution container 300 is inserted into the reaction receiving portion 404 of the reaction container 400. The third insertion portion 232 of the third cleaning container 230 is inserted into the elution receiving portion 304 of the elution container 300. The second insertion portion 222 of the second cleaning container 220 is inserted into the third receiving portion 234 of the third cleaning container 230. The first insertion portion 212 of the first cleaning container 210 is inserted into the second receiving portion 224 of the second cleaning container 220. The adsorption/insertion portion 122 of the adsorption container 100 is inserted into the first receiving portion 214 of the first cleaning container 210.

### (B) Process of Introducing Specimen

In the process of introducing the specimen, for example, a cotton applicator having the specimen attached thereto is introduced into the adsorption solution 10 through an opening on which the cap 110 of the adsorption container 100 is to be mounted, and is immersed in the adsorption solution 10. More specifically, the cotton applicator is introduced through the opening located in one end portion of the plunger portion 130 in a state of being inserted into the syringe portion 120 of the adsorption container 100. Next, the cotton applicator is detached from the adsorption container 100, and the cap 110 is mounted thereon. This process represents a state illustrated in FIG. 7A. The specimen may be introduced into the adsorption container 100 by using a pipette. If the specimen is in a paste state or in a solid state, the specimen may be placed into the adsorption container 100 by using a spoon or may be attached to the inner wall of the plunger portion 130 by using tweezers. As illustrated in FIG. 7A, the syringe portion 120 and the plunger portion 130 are intermediately filled with the adsorption solution 10. However, a space remains on the opening side on which the cap 110 is to be mounted.

The specimen contains the nucleic acid which serves as a target. Hereinafter, this is simply referred to as a target nucleic acid in some cases. For example, the target nucleic acid is deoxyribonucleic acid (DNA) and/or ribonucleic acid (RNA). The target nucleic acid is extracted from the specimen, is eluted in the eluent 32 (to be described later), and thereafter is utilized as a PCR template, for example. The specimen includes blood, nasal mucus, oral mucosa, and other various biological samples.

### (C) Process of Moving Magnetic Beads

The process of moving the magnetic beads 30 is performed by moving the magnet 3 toward the adsorption container 100 in a state where a magnetic force of the magnet 3 placed outside the container is applied to the magnetic beads 30 which are present in a plug state while being interposed between the third oil 24 and 24 of the second cleaning container 220 as illustrated in FIG. 7A.

In accordance with the movement of the magnetic beads 30 or prior to the movement, the cap 110 and the plunger portion 130 are moved in a direction in which both of these are withdrawn from the syringe portion 120, and the specimen inside the adsorption solution 10 is moved from the inside of the plunger portion 130 to the inside of the syringe portion 120. The movement of the plunger portion 130 causes the flow path 2 closed by the distal end portion 134 to communicate with the adsorption solution 10.

In accordance with the movement of the magnet 3, the magnetic beads 30 ascend inside the flow path 2, and reach the inside of the adsorption solution 10 in which the specimen is located, as illustrated in FIG. 7B.

### (D) Process of Causing Magnetic Beads to Adsorb Nucleic Acid

The process of adsorbing the nucleic acid is performed by shaking the adsorption container 100. This process can be efficiently performed, since the opening of the adsorption container 100 is sealed with the cap 110 so as to prevent the adsorption solution 10 from leaking out. This process allows the target nucleic acid to be adsorbed into the surface of the magnetic beads 30 by using an effect of a chaotropic agent. In this process, in addition to the target nucleic acid, other nucleic acid or proteins may be adsorbed into the surface of the magnetic beads 30.

As a method of shaking the adsorption container 100, a device such as a known vortex shaker may be used, or an operator's hand may be used in shaking the adsorption container 100. In addition, the adsorption container 100 may be shaken while a magnetic field is applied thereto from outside by utilizing magnetic properties of the magnetic beads 30.

### (E) Process of Moving Magnetic Beads Having Adsorbed Nucleic Acid

In the process of moving the magnetic beads 30 having the adsorbed nucleic acid, the magnetic beads 30 are moved while a magnetic force of the magnet 3 is applied thereto from the outside of the adsorption container 100, the cleaning container 200, and the elution container 300. In this manner, the magnetic beads 30 are moved through the adsorption solution 10, the first to fourth oil 20, 22, 24, and 26, and the first to third cleaning solutions 12, 14, and 16.

For example, as the magnet 3, a permanent magnet or an electromagnet can be used. The magnet 3 may be operated by an operator's hand, or may be operated by utilizing a mechanical device. The magnetic beads 30 have a property of being attracted by the magnetic force. Accordingly, this property is utilized so as to change a relative arrangement of the magnet 3 from the adsorption container 100, the cleaning container 200, and the elution container 300. In this manner, the magnetic beads 30 are moved inside the flow path 2. The speed of the magnetic beads 30 when passing through the respective cleaning solutions is not particularly limited. The magnetic beads 30 may be moved so as to reciprocate along the longitudinal direction of the flow path 2 inside the same cleaning solution. For example, when particles other than the magnetic beads 30 are intended to move inside a tube, the movement can be performed by utilizing gravity or a potential difference.

### (F) Process of Eluting Nucleic Acid

In the process of eluting the nucleic acid, the nucleic acid is eluted from the magnetic beads 30 toward the eluent droplet 36 inside the eluent container 300. The eluent 32 in FIGS. 7A and 7B is present as a plug in the thin portion of the flow path 2 in the elution container 300. However, while the magnetic beads 30 are moved as described above, the content solution is caused to expand by heating the reaction container 400. As illustrated in FIGS. 8A and 8B, the eluent 32 as the droplet 36 is moved upward inside the elution container 300. Then, as illustrated in FIG. 8A, if the magnetic beads 30 reach the eluent droplet 36 in the elution container 300, the target nucleic acid adsorbed into the magnetic beads 30 is eluted into the eluent droplet 36 by an effect of the eluent 32.

(G) Process of Bringing Droplet 36 into Contact with Reagent 34

In the process of bringing the droplet 36 into contact with the reagent 34, the droplet 36 containing the nucleic acid is brought into contact with the reagent 34 located in the lowest portion inside the reaction container 400. Specifically, as illustrated in FIG. 8B, the cap 110 is pressed, and the first oil 20 is pressed down by the distal end portion 134 of the plunger portion 130. In this manner, while the magnetic beads 30 to which the magnetic force of the magnet 3 is applied are maintained to remain at a predetermined position, the eluent droplet 36 having the eluted target nucleic acid is moved to the reaction container 400, and is brought into contact with the reagent 34 located in the lowest portion of the reaction container 400. The reagent 34 with which the droplet 36 comes into contact is melted and mixed with the target nucleic acid in the eluent. Therefore, for example, PCR using thermal cycle processing can be performed.

### 4. PCR Device

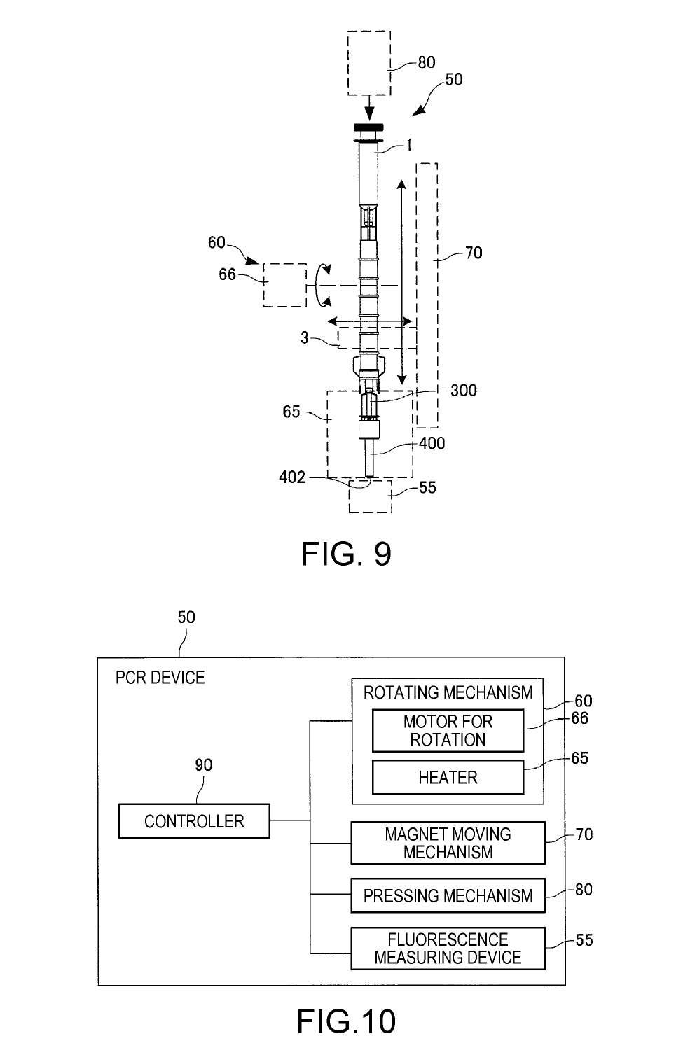

Referring to FIGS. 9 and 10, a PCR device 50 will be described which performs a nucleic acid eluting process and PCR by using the container assembly 1. FIG. 9 is a schematic configuration diagram of the PCR device 50. FIG. 10 is a block diagram of the PCR device 50.

The PCR device 50 has a rotating mechanism 60, a magnet moving mechanism 70, a pressing mechanism 80, a fluorescence measuring device 55, and a controller 90.

### 4-1. Rotating Mechanism

The rotating mechanism 60 includes a motor for rotation 66 and a heater 65. The container assembly 1 and the heater 65 are rotated by driving the motor for rotation 66. The rotating mechanism 60 rotates the container assembly 1 and the heater 65 so as to be turned upside down. In this manner, thermal cycle processing is performed by moving the droplet containing the target nucleic acid inside the flow path of the reaction container 400.

The heater 65 includes multiple heaters (not illustrated). For example, heaters for elution, high temperature, and low temperature can be included therein. The heater for elution heats the eluent in a plug state in the container assembly 1, thereby prompting the target nucleic acid to be eluted from the magnetic beads to the eluent. The heater for high temperature heats a liquid present on an upstream side of the flow path in the reaction container 400 by using a higher temperature than that of the heater for low temperature. The heater for low temperature heats the bottom portion 402 of the flow path in the reaction container 400. The heater for high temperature and the heater for low temperature can form a temperature gradient in the liquid inside the flow path in the reaction container 400. A temperature control device is disposed in the heater 65. In accordance with an instruction from the controller 90, the heater 65 can set the liquid inside the container assembly 1 to maintain a temperature suitable for processing.

The heater 65 has an opening through which an outer wall of the bottom portion 402 of the reaction container 400 is exposed. The fluorescence measuring device 55 measures brightness of an eluent droplet through the opening.

### 4-2. Magnet Moving Mechanism

The magnet moving mechanism 70 moves the magnet 3. The magnet moving mechanism 70 causes the magnet 3 to attract the magnetic beads inside the container assembly 1, and moves the magnet 3 so as to move the magnetic beads inside the container assembly 1. The magnet moving mechanism 70 has a pair of magnets 3, a lifting/lowering mechanism, and a shaking mechanism.

The shaking mechanism shakes the pair of magnets 3 in a lateral direction in FIG. 9 (may be a longitudinal direction in FIG. 9). The pair of magnets 3 are arranged so as to interpose the container assembly 1 mounted on the PCR device 50 therebetween in the lateral direction (refer to FIGS. 7A to 8B). The pair of magnets 3 can cause the magnetic beads and the magnet 3 to have a closer distance in a direction orthogonal to the flow path in the container assembly 1 (here, the lateral direction in FIG. 9). Therefore, if the pair of magnets 3 are shaken as illustrated by an arrow in the lateral direction, in accordance with the movement thereof, the magnetic beads inside the container assembly 1 are moved in the lateral direction. The lifting/lowering mechanism moves the magnet 3 in a vertical direction. In accordance with the movement of the magnet 3, the magnetic beads can be moved in the vertical direction in FIG. 9.

### 4-3. Pressing Mechanism

The pressing mechanism 80 presses the plunger portion of the container assembly 1. The plunger portion is pressed by the pressing mechanism 80, and the droplet inside the elution container 300 is pressed into the reaction container 400. In this manner, PCR can be performed inside the reaction container 400.

FIG. 9 illustrates the pressing mechanism 80 which is arranged above the upright container assembly 1. However, a direction in which the pressing mechanism 80 presses the plunger portion may be tilted by 45 degrees from the vertical direction, for example, instead of the vertical direction in FIG. 9. According to this tilting, it becomes easy to arrange the pressing mechanism 80 at a position where the pressing mechanism 80 does not interfere with the magnet moving mechanism 70.

### 4-4. Fluorescence Measuring Device

The fluorescence measuring device 55 measures brightness of the droplet in the reaction container 400. The fluorescence measuring device 55 is arranged at a position where the fluorescence measuring device 55 faces the bottom portion 402 of the reaction container 400. It is desirable that the fluorescence measuring device 55 can detect the brightness in multiple wavelength regions so as to be capable of corresponding to multiplex PCR.

### 4-5. Controller

The controller 90 controls the PCR device 50. For example, the controller 90 has a processor such as a CPU, and a storage device such as a ROM or a RAM. The storage device stores various programs and data. The storage device provides an area for program deployment. Various processes can be realized by causing the processor to execute the programs stored in the storage device.

For example, the controller 90 controls the motor for rotation 66 so as to rotate the container assembly 1 to reach a predetermined rotation position. A rotation position sensor (not illustrated) is disposed in the rotating mechanism 60. In accordance with the detection result of the rotation position sensor, the controller 90 drives or stops the motor for rotation 66.

The controller 90 controls the heater 65. The heater is heated through on/off control so as to heat the liquid inside the container assembly 1 up to a predetermined temperature.

The controller 90 controls the magnet moving mechanism 70 so as to move the magnet 3 in the vertical direction, and to shake the magnet 3 in the lateral direction in FIG. 9, in accordance with a detection result of a position sensor (not illustrated).

The controller 90 controls the fluorescence measuring device 55 so as to measure brightness of the droplet inside the reaction container 400. The measurement result is stored in a storage device (not illustrated) of the controller 90.

The container assembly 1 is mounted on the PCR device 50. In this manner, the above-described processes (C) to (G) in 3-2, and further the PCR can be performed.

### 5. Cartridge Set

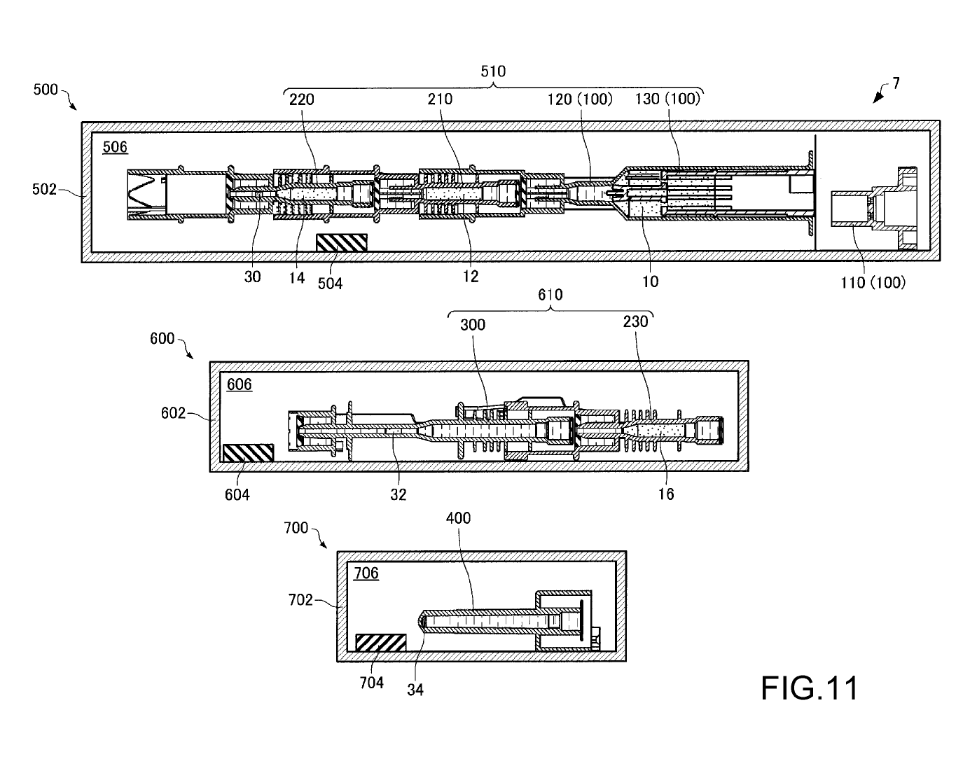

A cartridge set according to the embodiment will be described with reference to the drawings. FIG. 11 is a sectional view schematically illustrating a cartridge set 7 according to the embodiment. The above-described cartridge (container assembly) 1 can be obtained by assembling the cartridge set 7.

As illustrated in FIG. 11, the cartridge set 7 includes a first storage receptacle 500, a second storage receptacle 600, and a third storage receptacle 700. Hereinafter, the storage receptacles 500, 600, and 700 will be described.

### 5-1. First Storage Receptacle

As illustrated in FIG. 11, the first storage receptacle 500 includes a first package 502, a liquid holding member 504, the syringe portion 120 and the plunger portion 130 of the adsorption container 100, the first cleaning container 210, and the second cleaning container 220. In the illustrated example, the first storage receptacle 500 further includes the cap 110 of the adsorption container 100.

The first package 502 encloses and stores (hermetically seals) the liquid holding member 504, the adsorption container 100, and the cleaning containers 210 and 220. In the illustrated example, the first package 502 is illustrated as a bag-shaped package. However, a shape of the first package 502 is not particularly limited. For example, a box shape may be employed. A size of the first package 502 is not particularly limited as long as the liquid holding member 504, the adsorption container 100, and the cleaning containers 210 and 220 can be enclosed and stored therein.

Water permeability of the first package 502 is lower than water permeability of the adsorption container 100, and the cleaning containers 210 and 220. Here, the “water permeability” represents the amount of water (for example, water vapor) which passes through a package in a unit area per unit time at a predetermined temperature and humidity (which passes from the inner portion to the outside of the package or which passes from the outside to the inner portion of the package). More specifically, the “water permeability” represents permeability of water vapor, and may be determined based on JIS K7129.

Alcohol permeability of the first package 502 is lower than alcohol permeability of the adsorption container 100, and the cleaning containers 210 and 220. Here, the “alcohol permeability” is permeability with respect to alcohol, and represents the amount of alcohol (for example, gaseous alcohol) which passes through a package in a unit area per unit time at a predetermined temperature and humidity. For example, the expression of “the alcohol permeability is low” can also be replaced with the expression of “gas barrier properties are excellent”. The temperature required when the water permeability and the alcohol permeability are obtained is not particularly limited. For example, the temperature is 0° C. to 60° C., and is preferably room temperature. The water permeability and the alcohol permeability may be obtained depending on the thickness of the package.

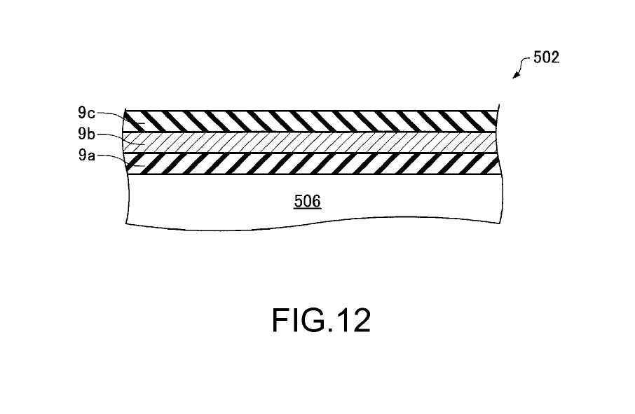

Preferably, a material of the first package 502 has low permeability of water vapor, and has excellent gas barrier properties. Specifically, the first package 502 is a bag having an aluminum layer. Here, FIG. 12 is a sectional view schematically illustrating the first package 502. As illustrated in FIG. 12, for example, the first package 502 has a polypropylene (PP) layer 9a, an aluminum layer 9b disposed on a surface of the PP layer 9a, and a polyethylene terephthalate (PET) layer 9c disposed on a surface of the aluminum layer 9b. In the illustrated example, the PP layer 9a side is an inner portion side of the first package 502, and the PET layer 9c side is an outer portion side of the first package 502. For example, two sheets to which the aluminum layer (aluminum foil) 9b is bonded by being interposed between the PP layer (PP film) 9a and the PET layer (PET film) 9c are prepared. The two sheets are superimposed on each other, and are thermally welded together so that the PP layers 9a come into contact with each other. In this manner, the first package 502 can be formed. The aluminum layer 9b may be formed by using a vacuum deposition method. The PP layer 9a and the PET layer 9c may be formed by using a film forming method such as an extrusion molding method.

Compared to polypropylene which is the material of the adsorption container 100 and the cleaning containers 210 and 220, aluminum has lower water permeability and lower alcohol permeability. Therefore, the first package 502 employs the bag having the aluminum layer 9b. In this manner, the water permeability of the first package 502 can be lower than the water permeability of the adsorption container 100 and the cleaning containers 210 and 220. Furthermore, the alcohol permeability of the first package 502 can be lower than the alcohol permeability of the adsorption container 100 and the cleaning containers 210 and 220. For example, the water permeability and the alcohol permeability of the first package 502 may be set to 0 g/m²·day (40° C., 90% RH).

The material of the first package 502 is not particularly limited as long as the first package 502 has lower water permeability and lower alcohol permeability than those of the adsorption container 100 and the cleaning containers 210 and 220. For example, instead of the aluminum layer 9b, a silica-deposited film may be used, or a layer including ethylene-vinyl alcohol copolymer resin may be used.

The liquid holding member 504 contains alcohol. A type of the alcohol contained in the liquid holding member 504 is the same as a type of the alcohol contained in the second cleaning solution 14 enclosed and stored in the second cleaning container 220. For example, the type is methanol, ethanol, propanol, acetonitrile, and isopropyl alcohol, and more preferably ethanol. Furthermore, when the adsorption solution 10 enclosed and stored in the adsorption container 100 contains the alcohol, the type of the alcohol contained in the liquid holding member 504 may be the same as the type of the alcohol contained in the adsorption solution 10. When the first cleaning solution 12 enclosed and stored in the first cleaning container 210 contains the alcohol, the type of the alcohol contained in the liquid holding member 504 may be the same as the type of the alcohol contained in the first cleaning solution 12. For example, the liquid holding member 504 may further contain water. The liquid holding member 504 can be maintained while the alcohol and the water are contained therein. For example, the liquid holding member 504 may be absorbent cotton soaked with alcohol and water (containing water and alcohol), or may be a porous body soaked with water and alcohol (specifically, a sponge).

The liquid holding member 504 can bring an inner portion 506 of the first package 502 into a state of being saturated with alcohol vapor. Furthermore, the liquid holding member 504 can bring the inner portion 506 into a state of being saturated with water vapor. Here, the “state of being saturated” represents a state where alcohol or water has saturated vapor pressure. Although not illustrated, the inner portion 506 may be brought into a state of being saturated with alcohol and water vapor by infusing liquid alcohol and water into the inner portion 506 without providing the liquid holding member 504.

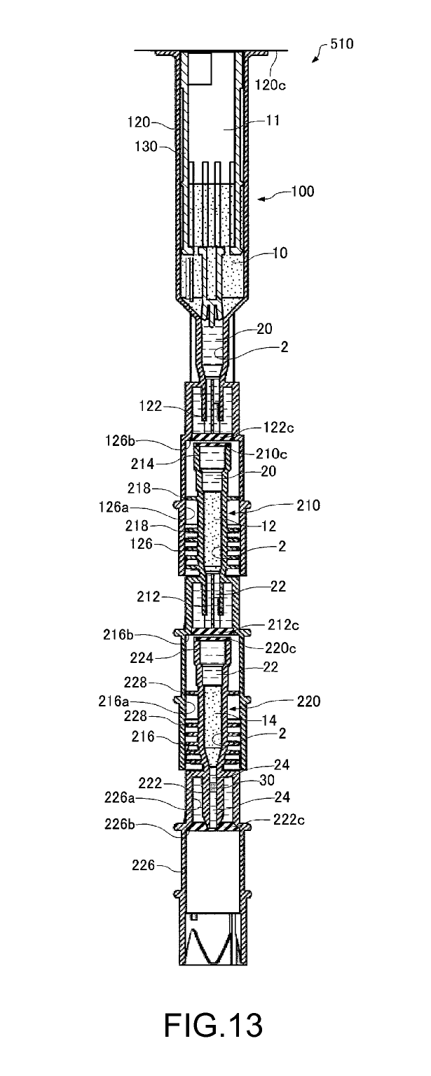

The syringe portion 120 and the plunger portion 130 of the adsorption container 100, and the cleaning containers 210 and 220 configure a first temporarily assembled body 510 in the inner portion 506 of the first package 502. Here, FIG. 13 is a sectional view schematically illustrating the first temporarily assembled body 510, and illustrates the same sectional view as that in FIG. 6.

In the first temporarily assembled body 510, as illustrated in FIG. 13, the flow path 2 of the adsorption container 100 and the flow path 2 of the first cleaning container 210 do not communicate with each other. Furthermore, in the first temporarily assembled body 510, the flow path 2 of first cleaning container 210 and the flow path 2 of the second cleaning container 220 do not communicate with each other.

In the first temporarily assembled body 510, the adsorption/insertion portion 122 of the adsorption container 100 is not inserted into the first receiving portion 214 of the first cleaning container 210. In the first temporarily assembled body 510, an inner wall 126a of the adsorption cover portion 126 is in contact with a flange 218 of the first cleaning container 210. Due to the friction between the adsorption cover portion 126 and the flange 218, the syringe portion 120 of the adsorption container 100 is temporarily fixed to the first cleaning container 210 in a state where the syringe portion 120 is less likely to move to the first cleaning container 210 in the vertical direction (longitudinal direction of the flow path 2).

The adsorption cover portion 126 is a portion which is formed around the adsorption/insertion portion 122 of the adsorption container 100, and which is open downward. The flange 218 is a portion which protrudes outward from the outer wall of the first cleaning container 210, and has an annular shape in a plan view.

A film 120c adheres to an upper end of the syringe portion 120 of the adsorption container 100. In the adsorption cover portion 126 of the adsorption container 100, an upper end thereof is connected to an outer wall of the adsorption/insertion portion 122, and a lower end thereof extends beyond the adsorption/insertion portion 122. The inner wall 126a of the adsorption cover portion 126 has an annular stepped portion 126b whose diameter increases downward. The stepped portion 126b is located slightly below a lower end of the adsorption/insertion portion 122, and a film 122c adheres to a surface thereof.

In the adsorption container 100, the films 120c and 122c enclose and store the adsorption solution 10 in which the nucleic acid is adsorbed into the nucleic acid binding activity solid-phase carrier (magnetic beads) 30, and a fluid (first oil) 20 which is immiscible with the adsorption solution 10. In the illustrated example, air 11, the adsorption solution 10, and the first oil 20 are sequentially arranged from the film 120c side toward the film 122c side. If the target nucleic acid is the RNA, for example, the adsorption solution 10 may contain alcohol (for example, ethanol), guanidine thiocyanate, and water. For example, the concentration of ethanol contained in the adsorption solution 10 may be 40 wt % to 50 wt %. If the target nucleic acid is the DNA, for example, the adsorption solution 10 may contain guanidine hydrochloride and water without containing ethanol or guanidine thiocyanate.

In the first temporarily assembled body 510, the first insertion portion 212 of the first cleaning container 210 is not inserted into the second receiving portion 224 of the second cleaning container 220. In the first temporarily assembled body 510, the inner wall 216a of the first cover portion 216 is in contact with a flange 228 of the second cleaning container 220. Due to the friction between the first cover portion 216 and the flange 228, the first cleaning container 210 is temporarily fixed to the second cleaning container 220 in a state where the first cleaning container 210 is less likely to move to the second cleaning container 220 in the vertical direction.

The first cover portion 216 is a portion which is formed around the first insertion portion 212 of the first cleaning container 210, and which is open downward. The flange 228 is a portion which protrudes outward from the outer wall of the second cleaning container 220, and has an annular shape in a plan view.

A film 210c adheres to an upper end of the first cleaning container 210. In the first cover portion 216 of the first cleaning container 210, an upper end thereof is connected to an outer wall of the first insertion portion 212, and a lower end thereof extends beyond the first insertion portion 212. The inner wall 216a of the first cover portion 216 has an annular stepped portion 216b whose diameter increases downward. The stepped portion 216b is located slightly below a lower end of the first insertion portion 212, and a film 212c adheres to a surface thereof.

In the first cleaning container 210, the films 210c and 212c enclose and store the first cleaning solution 12 for cleaning the magnetic beads 30 having the absorbed nucleic acid, and fluids (oil 20 and 22) which are immiscible with the first cleaning solution 12. In the illustrated example, the first oil 20, the first cleaning solution 12, and the second oil 22 are sequentially arranged from the film 210c side toward the film 212c side. If the target nucleic acid is the RNA, for example, the first cleaning solution 12 may contain alcohol (for example, ethanol), guanidine hydrochloride, and water. For example, the concentration of ethanol contained in the first cleaning solution 12 may be 50 wt % to 60 wt %. If the target nucleic acid is the DNA, for example, the first cleaning solution 12 may contain guanidine hydrochloride and water without containing ethanol.

In the first temporarily assembled body 510, a film 220c adheres to an upper end of the second cleaning container 220. In the second cover portion 226 of the second cleaning container 220, an upper end thereof is connected to an outer wall of the second insertion portion 222, and a lower end thereof extends beyond the second insertion portion 222. The inner wall 226a of the second cover portion 226 has an annular stepped portion 226b whose diameter increases downward. The stepped portion 226b is located slightly below a lower end of the second insertion portion 222, and a film 222c adheres to a surface thereof.

In the second cleaning container 220, the films 220c and 222c enclose and store the second cleaning solution 14 for cleaning the magnetic beads 30 having the absorbed nucleic acid, fluids (oil 22 and 24) which are immiscible with the second cleaning solution 14, and the magnetic beads 30. In the illustrated example, the second oil 22, the second cleaning solution 14, the third oil 24, the magnetic beads 30, and the third oil 24 are sequentially arranged from the film 220c side toward the film 222c side. If the target nucleic acid is the RNA, for example, the second cleaning solution 14 may contain alcohol (for example, ethanol), sodium chloride, and water. For example, the concentration of ethanol contained in the second cleaning solution 14 may be 60 wt % to 70 wt %. If the target nucleic acid is the DNA, for example, the second cleaning solution 14 may contain ethanol and water without containing sodium chloride.

### 5-2. Second Storage Receptacle

As illustrated in FIG. 11, the second storage receptacle 600 includes a second package 602, a liquid holding member 604, the third cleaning container 230, and the elution container 300.

The second package 602 encloses and stores (hermetically seals) the liquid holding member 604, the third cleaning container 230, and the elution container 300. A shape of the second package 602 is not particularly limited, and may be the bag shape or the box shape similar to the first package 502. A size of the second package 602 is not particularly limited as long as the liquid holding member 604, the third cleaning container 230, and the elution container 300 can be enclosed and stored therein. For example, the second package 602 is formed by using the same method as that of the first package 502.

Water permeability of the second package 602 is lower than water permeability of the third cleaning container 230 and the elution container 300. Alcohol permeability of the second package 602 is lower than alcohol permeability of the third cleaning container 230 and the elution container 300. For example, similarly to the first package 502, the second package 602 is a bag having an aluminum layer. For example, the water permeability and the alcohol permeability of the second package 602 may be set to 0 g/m²·day (40° C., 90% RH).

The liquid holding member 604 contains water. The liquid holding member 604 can be maintained while the water is contained therein. The liquid holding member 604 may be absorbent cotton soaked with water (containing water), or may be a porous body soaked with water (specifically, a sponge). The liquid holding member 604 can bring an inner portion 606 of the second package 602 into a state of being saturated with water vapor. Although not illustrated, the inner portion 606 may be brought into a state of being saturated with water vapor by infusing liquid water into the inner portion 606 without providing the liquid holding member 604.

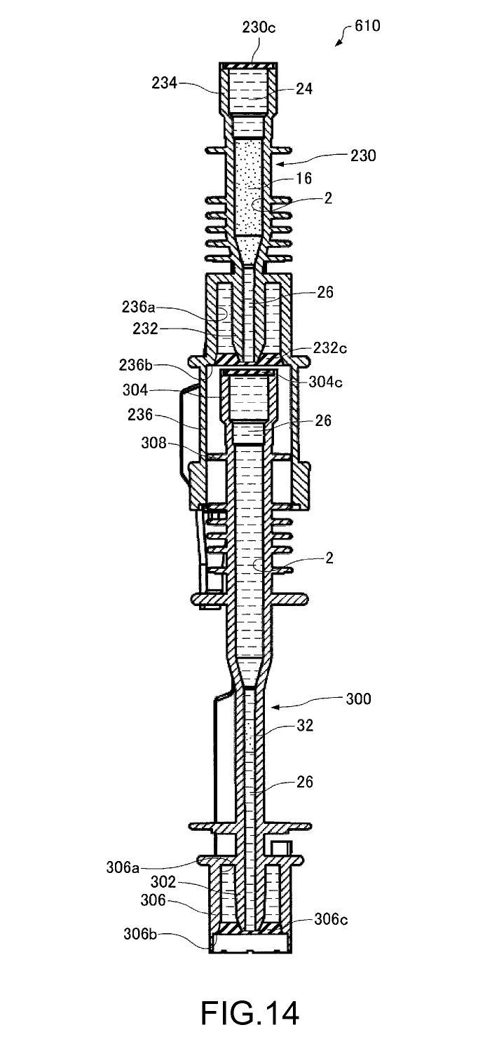

The third cleaning container 230 and the elution container 300 configure a second temporarily assembled body 610 in the inner portion 606 of the second package 602. Here, FIG. 14 is a sectional view schematically illustrating the second temporarily assembled body 610, and illustrates the same sectional view as that in FIG. 6.

In the second temporarily assembled body 610, as illustrated in FIG. 14, the flow path 2 of the third cleaning container 230 and the flow path 2 of the elution container 300 do not communicate with each other. In the second temporarily assembled body 610, the third insertion portion 232 of the third cleaning container 230 is not inserted into the elution receiving portion 304 of the elution container 300. In the second temporarily assembled body 610, the inner wall 236a of the third cover portion 236 is in contact with a flange 308 of the elution container 300. Due to the friction between the third cover portion 236 and the flange 308, the third cleaning container 230 is temporarily fixed to the elution container 300 in a state where the third cleaning container 230 is less likely to move to the elution container 300 in the vertical direction.

The third cover portion 236 is a portion which is formed around the third insertion portion 232 of the third cleaning container 230, and which is open downward. The flange 308 is a portion which protrudes outward from the outer wall of the elution container 300, and has an annular shape in a plan view.

A film 230c adheres to an upper end of the third cleaning container 230. In the third cover portion 236 of the third cleaning container 230, an upper end thereof is connected to an outer wall of the third insertion portion 232, and a lower end thereof extends beyond the third insertion portion 232. The inner wall 236a of the third cover portion 236 has an annular stepped portion 236b whose diameter increases downward. The stepped portion 236b is located slightly below a lower end of the third insertion portion 232, and a film 232c adheres to a surface thereof.

In the third cleaning container 230, the films 230c and 232c enclose and store the third cleaning solution 16 for cleaning the magnetic beads 30 having the absorbed nucleic acid, and fluids (oil 24 and 26) which are immiscible with the third cleaning solution 16. In the illustrated example, the third oil 24, the third cleaning solution 16, and the fourth oil 26 are sequentially arranged from the film 230c side toward the film 232c side. For example, the third cleaning solution 16 may contain citric acid and water. The third cleaning solution 16 does not contain alcohol.

A film 304c adheres to an upper end of the elution container 300. In the elution cover portion 306 of the elution container 300, an upper end thereof is connected to an outer wall of the elution/insertion portion 302, and a lower end thereof extends beyond the elution/insertion portion 302. The inner wall 306a of the elution cover portion 306 has an annular stepped portion 306b whose diameter increases downward. The stepped portion 306b is located slightly below a lower end of the elution/insertion portion 302, and a film 306c adheres to a surface thereof.

In the elution container 300, the films 304c and 306c enclose and store the eluent 32 for eluting the nucleic acid from the magnetic beads 30, and a fluid (fourth oil 26) which is immiscible with the eluent 32. In the illustrated example, the fourth oil 26, the eluent 32, and the fourth oil 26 are sequentially arranged from the film 304c side toward the film 306c side. For example, the eluent 32 may contain water.

### 5-3. Third Storage Receptacle

As illustrated in FIG. 11, the third storage receptacle 700 includes a third package 702, a drying agent 704, and the reaction container 400.

The third package 702 encloses and stores (hermetically seals) the drying agent 704 and the reaction container 400. A shape of the third package 702 is not particularly limited, and may be the bag shape or the box shape similar to the first package 502. A size of the third package 702 is not particularly limited as long as the drying agent 704 and the reaction container 400 can be enclosed and stored therein. For example, the third package 702 is formed by using the same method as that of the first package 502. In the illustrated example, the packages 502, 602, and 702 are separated from each other. Respective volumes of the inner portions 506, 606, and 706 of the packages 502, 602, and 702 may be different from each other, or may be the same as each other.

Water permeability of the third package 702 is lower than water permeability of the reaction container 400. Alcohol permeability of the third package 702 is lower than alcohol permeability of the reaction container 400. For example, similarly to the first package 502, the third package 702 is a bag having an aluminum layer. For example, the water permeability and the alcohol permeability of the third package 702 may be set to 0 g/m²·day (40° C., 90% RH) .

For example, the drying agent 704 is a molecular sieve or silica gel. However, in view of water absorption at low humidity, it is preferable to use the molecular sieve. The molecular sieve is a crystalline zeolite, and is a crystalline material of an aluminosilicate material. The drying agent 704 absorbs water in the inner portion 706 of the third package 702.

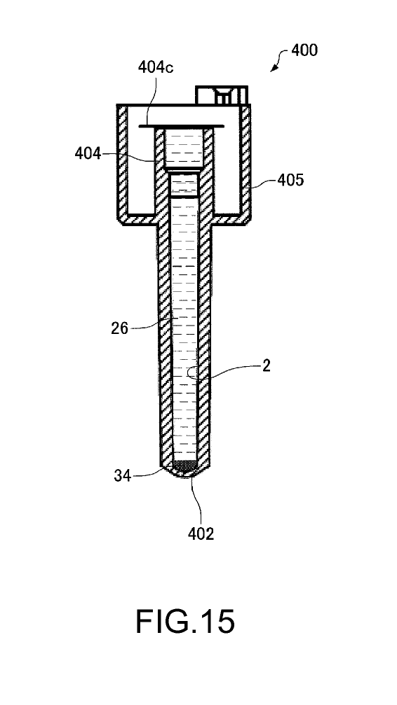

Here, FIG. 15 is a sectional view schematically illustrating the reaction container 400 in the inner portion 706 of the third package 702, and illustrates the same sectional view as that in FIG. 6.

As illustrated in FIG. 15, the reaction container 400 has a reaction cover portion 405 which is formed around the reaction receiving portion 404, and which is open upward. In the reaction cover portion 405, a lower end thereof is connected to an outer wall of the reaction receiving portion 404, and an upper portion thereof extends beyond the reaction receiving portion 404. A film 404c adheres to an upper surface of the reaction receiving portion 404.

In the reaction container 400, the film 404c encloses and stores the reagent 34 for performing nucleic acid amplification reaction (for performing PCR), and a fluid (fourth oil 26) which is immiscible with the reagent 34. In the illustrated example, the reagent 34 is disposed in the bottom portion 402 of the reaction container 400. For example, the reagent 34 may be lyophilized (freeze-dried). Specifically, the reagent 34 is obtained by being rapidly frozen at approximately −80° C. and being further dried after moisture sublimation in a decompressed state. For example, the fourth oil 26 may be oil dehydrated by a molecular sieve. For example, the reagent 34 may be arranged inside the fourth oil 26.

### 5-4 Assembling Method

An example of an assembling method for the cartridge set 7 will be described. First, the first temporarily assembled body 510 (refer to FIGS. 11 and 13) is detached from the first package 502. Then, the adsorption/insertion portion 122 of the adsorption container 100 is inserted into the first receiving portion 214 of the first cleaning container 210, thereby joining the adsorption container 100 and the first cleaning container 210 to each other. The films 122c and 210c are broken by the adsorption/insertion portion 122 and the first receiving portion 214. This allows the flow path 2 of the adsorption container 100 and the flow path 2 of the first cleaning container 210 to communicate with each other. For example, the film 120c is broken when a cotton applicator having a specimen attached thereto is introduced into the adsorption solution 10 through the opening on which the cap 110 of the adsorption container 100 is to be mounted.

Next, the first insertion portion 212 of the first cleaning container 210 is inserted into the second receiving portion 224 of the second cleaning container 220, thereby joining the first cleaning container 210 and the second cleaning container 220 to each other. The films 212c and 220c are broken by the first insertion portion 212 and the second receiving portion 224. This allows the flow path 2 of the first cleaning container 210 and the flow path 2 of the second cleaning container 220 to communicate with each other.

Next, the second temporarily assembled body 610 (refer to FIGS. 11 and 14) is detached from the second package 602. Then, the third insertion portion 232 of the third cleaning container 230 is inserted into the elution receiving portion 304 of the elution container 300, thereby joining the third cleaning container 230 and the elution container 300 to each other. The films 232c and 304c are broken by the third insertion portion 232 and the elution receiving portion 304. This allows the flow path 2 of the third cleaning container 230 and the flow path 2 of the elution container 300 to communicate with each other. In this manner, the respective flow paths 2 from the adsorption container 100 to the elution container 300 are allowed to communicate with each other.

Next, the second insertion portion 222 of the second cleaning container 220 is inserted into the third receiving portion 234 of the third cleaning container 230, thereby joining the second cleaning container 220 and the third cleaning container 230 to each other. The films 222c and 230c are broken by the second insertion portion 222 and the third receiving portion 234. This allows the flow path 2 of the second cleaning container 220 and the flow path 2 of the third cleaning container 230 to communicate with each other. In this manner, the respective flow paths 2 from the adsorption container 100 to the reaction container 400 are allowed to communicate with each other.

Next, the reaction container 400 (refer to FIGS. 11 and 15) is detached from the third package 702. Then, the elution/insertion portion 302 of the elution container 300 is inserted into the reaction receiving portion 404 of the reaction container 400, thereby joining the elution container 300 and the reaction container 400 to each other. The films 306c and 404c are broken by the elution/insertion portion 302 and the reaction receiving portion 404. This allows the flow path 2 of the elution container 300 and the flow path 2 of the reaction container 400 to communicate with each other.

Through the above-described processes, the cartridge 1 (refer to FIGS. 5 and 6) can be obtained by assembling the cartridge set 7. An order for joining the respective containers 100, 210, 220, 230, 300, and 400 is not limited to the above-described example. An order for detaching the respective containers 100, 210, 220, 230, 300, and 400 from the respective packages 502, 602, and 702 is also not limited to the above-described example.

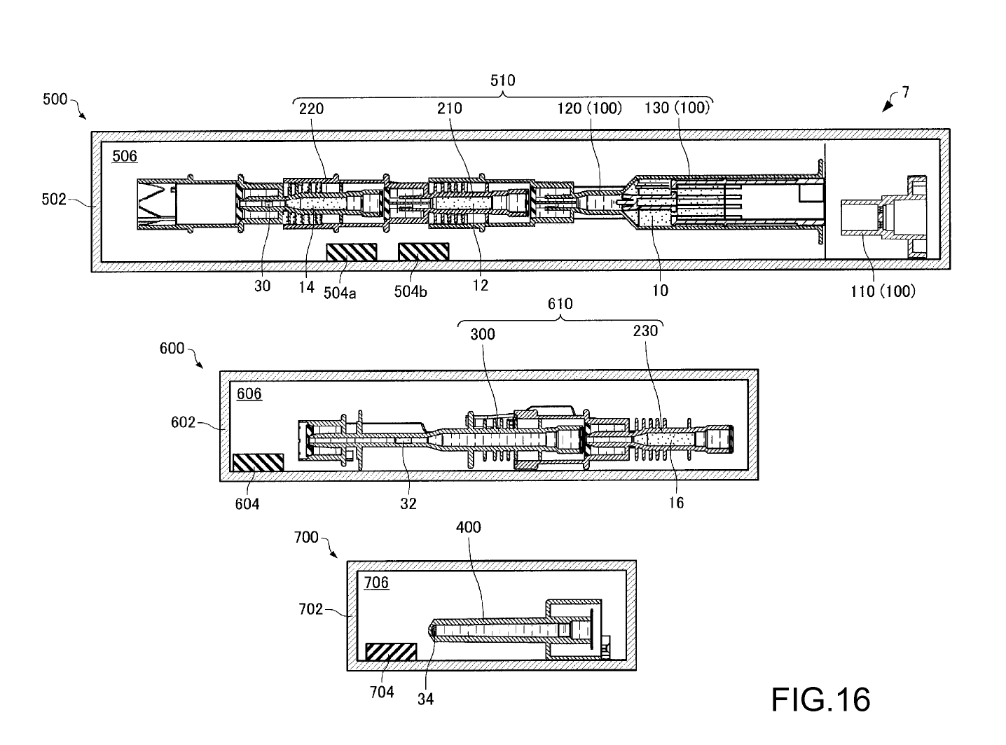

In the above description, an example has been described in which a single liquid holding member 504 is enclosed and stored in the first package 502. However, as illustrated in FIG. 16, two liquid holding members 504a and 504b may be enclosed and stored in the first package 502. The liquid holding member 504a may be absorbent cotton containing alcohol. The liquid holding member 504b may be absorbent cotton containing water.

In the above description, an example has been described in which in the first package 502, the adsorption container 100 and the cleaning containers 210 and 220 configure the first temporarily assembled body 510, and in which the third cleaning container 230 and the elution container 300 configure the second temporarily assembled body 610. However, the containers 100, 210, 220, 230, and 300 may be separated from each other without configuring any temporarily assembled body (not illustrated).

A second storage receptacle (container storage receptacle) 600 or the cartridge set 7 has the following features, for example.

The container storage receptacle 600 includes the elution container 300 which is enclosed and stored in the second package 602 and in which the eluent 32 is enclosed and stored. The eluent 32 contains water, and the inner portion 606 of the second package 602 is in a state of being saturated with water vapor. Therefore, according to the container storage receptacle 600, water contained in the eluent 32 inside the elution container 300 can be prevented from evaporating after permeating through the elution container 300 and the second package 602. Therefore, according to the container storage receptacle 600, even in a case of long-term storage, the eluent 32 can be prevented from evaporating. Furthermore, according to the container storage receptacle 600, for example, moisture in the atmosphere can be prevented from entering the inside of the elution container 300.

For example, if the eluent evaporates, the amount of the eluent decreases, thereby causing difficulties in forming a plug between the eluent and the oil in some cases. In particular, a small amount of the eluent is used. Accordingly, if the eluent partially evaporates, it becomes difficult to form the plug. Furthermore, concentration of the reagent in PCR is sometimes forced to increase. As a result, the PCR is adversely affected in some cases.

For example, if moisture in the atmosphere enters the inside of the elution container, the amount of the eluent increases. When thermal cycle processing is performed in the PCR by rotating a cartridge 1, a droplet containing a target nucleic acid can no longer smoothly move in some cases. As a result, the PCR is adversely affected in some cases.

The container storage receptacle 600 includes the liquid holding member 604 which is enclosed and stored in the second package 602, and which contains water. Therefore, according to the container storage receptacle 600, the inner portion 606 of the second package 602 can be brought into a state of being saturated with water vapor without infusing liquid water into the second package 602. For example, if the liquid water is infused into the second package, when the second temporarily assembled body is detached from the second package, water spills therefrom in some cases. Therefore, according to container storage receptacle 600, it is not necessary to infuse the liquid water into the second package 602. Accordingly, when the third cleaning container 230 and the elution container 300 are detached from the second package 602, there is no possibility that the water may spill therefrom.

In the container storage receptacle 600, water permeability of the second package 602 is lower than water permeability of the elution container 300. Therefore, according to the container storage receptacle 600, water contained in the eluent 32 inside the elution container 300 can be more reliably prevented from evaporating after permeating through the elution container 300 and the second package 602.

The container storage receptacle 600 includes the third cleaning container 230 which is enclosed and stored in the second package 602 and in which the third cleaning solution 16 is enclosed and stored. Water permeability of the second package 602 is lower than water permeability of the third cleaning container 230. Therefore, according to the container storage receptacle 600, the water contained in the third cleaning solution 16 inside the third cleaning container 230 can be prevented from evaporating after permeating through the second package 602.

For example, if water contained in the third cleaning solution evaporates, in some cases, air enters the inside of the third cleaning container, thereby generating bubbles in the flow path of the third cleaning container. Then, when the magnetic beads having the adsorbed nucleic acid are moved, the magnetic beads are trapped at an interface of the bubbles in some cases. The cases depend on a diameter of the flow path in the third cleaning container. For example, if alcohol or water of 0.8 μl (micro liters) evaporates, the flow path is blocked by the generated bubbles in some cases. As a result, PCR is adversely affected in some cases.

In the container storage receptacle 600, the second package 602 is a bag having the aluminum layer 9b. Therefore, according to the container storage receptacle 600, the water permeability of the second package 602 can be lowered.

The cartridge set 7 includes the second cleaning container 220 which is enclosed and stored in the first package 502, and in which the second cleaning solution 14 is enclosed and stored, the elution container 300 which is enclosed and stored in the second package 602, and in which the eluent 32 is enclosed and stored, and the reaction container 400 which is enclosed and stored in the third package 702, and in which the reagent 34 is enclosed and stored. Water permeability of the first package 502 is lower than water permeability of the second cleaning container 220, water permeability of the second package 602 is lower than water permeability of the elution container 300, and water permeability of the third package 702 is lower than water permeability of the reaction container 400. Therefore, according to the cartridge set 7, compared to a case where each container is not enclosed and not stored in each package, it is possible to further prevent water from moving between the containers (for example, movement of water between the containers 220 and 400 or movement of water between the containers 300 and 400). In particular, according to the cartridge set 7, water contained in the second cleaning solution 14 or in the eluent 32 can be prevented from coming into contact with the reagent 34 after entering the inside of the reaction container 400. Therefore, according to the cartridge set 7, even in a case of long-term storage, the water can be prevented from coming into contact with the reagent 34.

Furthermore, the cartridge set 7 encloses and stores the first temporarily assembled body 510, the second temporarily assembled body 610, and the reaction container 400 in respectively separated packages. Therefore, according to the cartridge set 7, even in a case of long-term storage, water contained in the second cleaning solution 14 or in the eluent 32 can be prevented from coming into contact with the reagent 34 through the flow path 2.

For example, if water comes into contact with a lyophilized reagent, enzymes contained in the reagent are sometimes degraded in a short period of time. Furthermore, if water comes into contact with a reagent for performing a nucleic acid amplification reaction, the reagent becomes glutinous (viscosity of the reagent becomes higher) in some cases. When a droplet containing the nucleic acid comes into contact with the reagent, the reagent becomes less soluble, and thus the nucleic acid and the reagent are less likely to be mixed together in a solution in some cases. As a result, PCR (nucleic acid amplification reaction) is inhibited in some cases. For example, if water of approximately 0.1 mass % per reagent for performing a nucleic acid amplification reaction comes into contact with the reagent, PCR is inhibited.

In the cartridge set 7, the inner portion 506 of the first package 502 is in a state of being saturated with alcohol vapor and water vapor. Therefore, according to the cartridge set 7, for example, alcohol or water contained in the second cleaning solution 14 inside the second cleaning container 220 can be prevented from evaporating after permeating through the second cleaning container 220 and the first package 502. Similarly, the adsorption solution 10 inside the adsorption container 100 and the first cleaning solution 12 inside the first cleaning container 210 can also be prevented from evaporating.

The cartridge set 7 includes the drying agent 704 which is enclosed and stored in the third package 702. Therefore, according to the cartridge set 7, for example, even if water vapor in the atmosphere enters the inside of the third package 702, the drying agent 704 absorbs the water vapor. Accordingly, the water vapor can be prevented from coming into contact with the reagent 34.

### 6. Modification Example of Cartridge Set

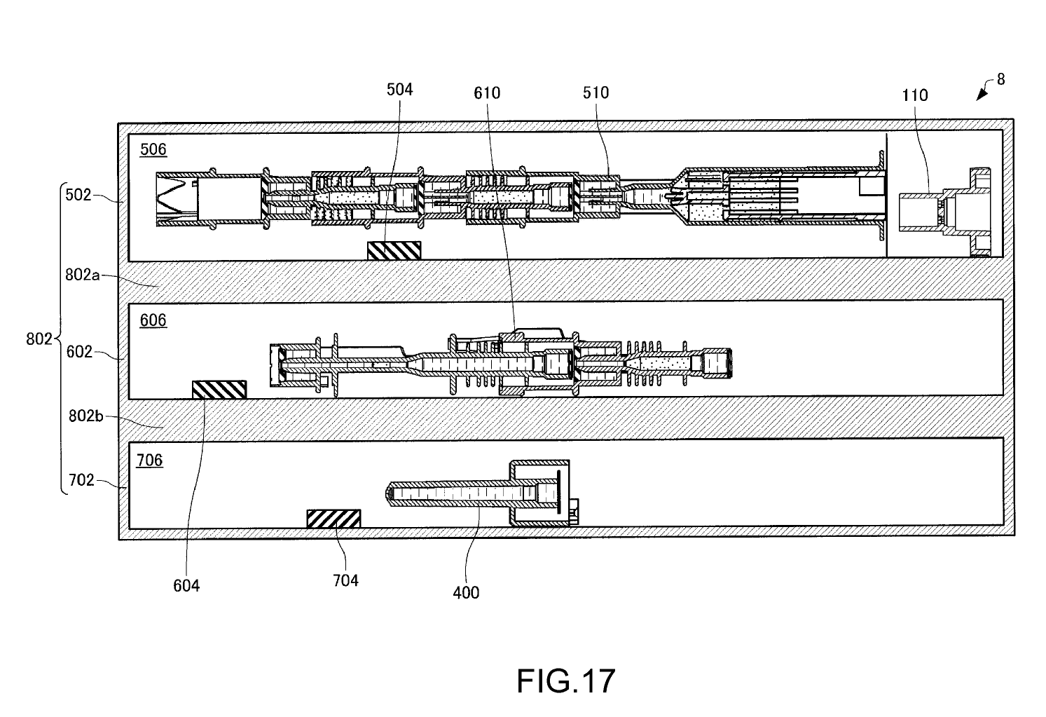

A cartridge set according to a modification example of the embodiment will be described with reference to the drawing. FIG. 17 is a sectional view schematically illustrating a cartridge set 8 according to the modification example of the embodiment. Hereinafter, the cartridge set 8 according to the modification example of the embodiment will be described focusing on points which are different from those of the cartridge set 7 according to the embodiment, and description on the same points will be omitted.

In the above-described cartridge set 7, as illustrated in FIG. 11, the packages 502, 602, and 702 are separated from each other. In contrast, in the cartridge set 8, as illustrated in FIG. 17, the packages 502, 602, and 702 are continuous with each other.

Specifically, according to the cartridge set 8, a first welding portion 802a and a second welding portion 802b are formed by thermally welding a large package 802, thereby continuously forming the packages 502, 602, and 702. The temporarily assembled bodies 510 and 610 and the reaction container 400 are respectively stored in the packages 502, 602, and 702 so as to extend along the longitudinal direction of the flow path 2. For example, respective volumes of the inner portions 506, 606, and 706 of the packages 502, 602, and 702 are the same as each other. In the illustrated example, an arrangement is made so that the second package 602 is located between the first package 502 and the third package 702. However, each arrangement location of the packages 502, 602, and 702 is not particularly limited.

In the cartridge set 8, the packages 502, 602, and 702 are continuous with each other. Accordingly, the temporarily assembled bodies 510 and 610 and the reaction container 400 can be easily detached from the respective packages 502, 602, and 702. For example, according to the cartridge set 7, in order to detach the temporarily assembled bodies 510 and 610 and the reaction container 400, it is necessary to tear the packages 502, 602, and 702 once each, thrice in total. According to the cartridge set 8, the temporarily assembled bodies 510 and 610 and the reaction container 400 can be easily detached by tearing the large package 802 only once.

Without being limited to the above-described embodiments, the invention may be further modified in various ways. For example, the invention includes configurations which are substantially the same as the configurations described in the embodiments (for example, the same configurations having the same function, method, and result, or the same configurations having the same object and advantageous effect). The invention includes configurations which replace non-essential elements of the configurations described in the embodiments. The invention includes configuration which can provide operation effects the same as those of the configurations described in the embodiments, or configurations which can achieve the same object. The invention includes configurations in which known techniques are added to the configurations described in the embodiments.

The entire disclosure of Japanese Patent Application No. 2014-232505, filed Nov. 17, 2014 is expressly incorporated by reference herein.

What is claimed is:

1. A container storage receptacle comprising:
a package; and
an elution container that is enclosed and stored in the package, and that encloses and stores an eluent for eluting a nucleic acid from a nucleic acid binding activity solid-phase carrier,
wherein the eluent contains water, and an inner portion of the package is in a state of being saturated with water vapor.

2. The container storage receptacle according to claim 1, further comprising:
a liquid holding member that is enclosed and stored in the package, and that contains water.

3. The container storage receptacle according to claim 2,
wherein the liquid holding member is absorbent cotton.

4. The container storage receptacle according to claim 1,
wherein water permeability of the package is lower than water permeability of the elution container.

5. The container storage receptacle according to claim 1,
wherein a fluid which is immiscible with the eluent is enclosed and contained in the elution container.

6. The container storage receptacle according to claim 1, further comprising:
a cleaning container that is enclosed and stored in the package, and that encloses and stores a cleaning solution for cleaning a nucleic acid binding activity solid-phase carrier having the adsorbed nucleic acid,
wherein the cleaning solution contains the water, and water permeability of the package is lower than water permeability of the cleaning container.

7. The container storage receptacle according to claim 1,
wherein the package is a bag which has an aluminum layer.
